(19) United States
(12) Patent Application Publication
(10) Pub. No.: US 2011/0206642 A1
(43) Pub. Date: Aug. 25, 2011

(54) Preventing obesity-related metabolic syndrome with melagonesis

(75) Inventor: Ancha Baranova, Annandale, VA (US)

(73) Assignee: Cohava Gelber, Nokesville, VA (US)

(21) Appl. No.: 12/925,177

(22) Filed: Oct. 14, 2010

Related U.S. Application Data
(60) Provisional application No. 61/251,737, filed on Oct. 15, 2009.

(51) Int. Cl. A61K 35/74; A61K 38/34; A61P 3/04; A61P 9/00; A61P 3/10

(52) U.S. Cl. 424/93.4; 514/10.7

(57) ABSTRACT

Anti-inflammatory effect of α-MSH-like compounds can be employed for curtailing sequelae of obesity and overweight. Importantly, molecular compounds that stimulate melanin biosynthesis by mimicking the effects of α-MSH are readily available for trials aimed at control of metabolic syndrome components. Synthetic agonists of α-MSH receptors, melanotan II and bremelanotide, have already been proven safe in human trials for therapeutic tanning and some non-obesity-related diseases.

The abatement of the non-communicable age-related diseases (NCDs) such as heart disease, cancer, stroke, type 2 diabetes and chronic lower respiratory diseases is a global challenge assigned high priority by The World Health Organization (WHO) [1]. Tobacco smoking, physical inactivity and the resulting obesity are established risk factors for many NCDs. The pathogenesis of NCDs is complex. Moreover, each chronic illness of NCD type cannot be considered in isolation as they share common, usually related risk factors [2]. This observation indicates that integrated strategies can be effective for many different conditions [3].

One common factor that initiates or hastens progression of almost all NCDs is a systemic low-grade inflammation. Both chronic inflammation and reactive oxygen species (ROS) are also key features of ageing. The interaction between inflammatory and insulin/IGF-1 signaling pathways is well established; in that, subclinical inflammation increases insulin resistance. In overweight and obese populations prevalent in the US, the subclinical inflammation propagates by the excessive adipocytic production of the pro-inflammatory cytokines including TNF-α and IL-6, possibly compounded by the infiltration of adipose by macrophages [6]. In turn, the cytokine production by adipocytes and macrophages contributes to obesity-related insulin resistance, thus, propagating the greatest vicious circle of NCDs.

### CROSS-REFERENCE TO RELATED APPLICATIONS

This application claims priority to U.S. Provisional Patent Application No. 61/251,737 filed Oct. 14, 2009, entitled “Preventing Obesity-related Metabolic Syndrome with Melanogenesis”, and said provisional patent application is hereby incorporated by reference in its entirety, including all tables, figures and claims.”

### FIELD OF INVENTION

The present invention relates to α-MSH and α-MSH related compounds that can be employed for curtailing inflammation various disease situations including secondary complications of metabolic syndromes.

### BACKGROUND OF INVENTION

Over the past 20 years, obesity has become pandemic. Currently, 127 million (66.3%) adults in the U.S. are overweight, 60 million (32.2%) are obese, and 9 million (4.8%) are severely obese (Ogden LC et al., 2006). Obesity often leads to the development of several chronic conditions, including type II diabetes, cardiovascular disease, and nonalcoholic fatty liver disease, all of which result in high medical costs (Wild S H, Byrne C D, 2006; Caballería L et al., 2008). However, the effects of body weight on morbidity and mortality are highly individual. A substantial number of the morbidly obese patients with BMIs>45 remain relatively healthy and have normal sensitivity to insulin (Nakamuta M et al., 2008; Jarrar et al., 2008).

Others may develop insulin resistance and subsequent type II diabetes while being merely overweight (Nakamuta M et al., 2008). This variance between individuals is routinely explained by gene-environment interactions (Andreassi MG, 2009; Qi L, and Cho Y A, 2008; Romao I and Roth J, 2008). Unraveling the details of these interactions is a complex task that is far from being completed.

Studies of metabolic syndrome, a cluster of risk factors predisposing an individual to obesity-associated morbidity, recently redefined obesity as a chronic inflammatory state that advances with increased production of pro-inflammatory cytokines and adipokines by the growing adipocytic mass (Nathan C et al., 2008; de Luca C and Olefsky J M, 2008). Nevertheless, in a subset of morbidly obese people, the adipose tissue remains relatively inert, sustaining low levels of secretion and resisting the development of metabolic syndrome (Jarrar et al., 2008). Currently, there is no explanation for this phenomenon.

The melanocortin system controls the pigmentation of skin and hair by stimulating melanin biosynthesis. In humans, melanin is produced exclusively in melanocytes, in retinal pigment epithelium (RPE) cells, and in cells of the inner ear and the central nervous system. Our recent study demonstrated for the first time that the melanin biosynthesis pathway functions in adipose tissue and is hyperactive in visceral fat samples of morbidly obese individuals [Randhawa et al., 2009]. Both the causes and the consequences of melanin production in adipose tissue remain unknown.

We propose the connection between extra-cutaneous accumulations of melanin and the delay of in the development of the sequelae of morbid obesity. This connection has never been described before, although one type of hyperpigmentation of the skin, known as acanthosis nigricans, has been associated with either clinical or subclinical insulin resistance, a component of metabolic syndrome [Guran T. et al, 2008]. We propose that the ectopic synthesis of melanin may serve as a compensatory mechanism that utilizes melanin's anti-inflammatory (Mohagheghpour N et al., 2000) and oxidative damage-absorbing (Rózanowska M et al., 1999; Seagle B L et al., 2005) properties. As obesity progresses and cellular fat deposition increases, adipocytes become more exposed to endogenous apoptotic signals, especially ROS. To counteract pro-apoptotic ROS effects, the adipocytes may, in turn, ectopically activate the genetic program of melanogenesis, thus neutralizing excessive ROS. Adipocytic melanin would also suppress the secretion of proinflammatory molecules (Mohagheghpour N et al., 2000), thereby decreasing the pro-inflammatory background in obese subjects and alleviating the metabolic syndrome. High levels of polymorphisms in human genes regulating melanin biosynthesis may account for the highly individual melanogenic responses of adipocytes and for the differences in an individual's propensity to develop secondary complications of obesity.

### SUMMARY OF THE INVENTION

While studying visceral adipose, we discovered the presence of intracellular melanin and expression of melanogenesis-related genes in human adipocytes. Moreover, when visceral fat of morbidly obese individuals was compared to that of lean subjects, these genes were found to be expressed at higher levels. This increase was paralleled by an increase in eumelanin content. What could plausibly explain the fact that the adipose tissue of morbidly obese patients produces higher levels of melanin than that of lean subjects? We hypothesized that ectopic synthesis of the melanin serves as a compensatory mechanism that utilizes the anti-inflammatory and oxidative damage-absorbing abilities of this compound. With the progression of obesity and an increase in the cellular fat depot, adipocytes become more exposed to endogenous apoptotic signals, especially reactive oxygen species (ROS). To counteract pro-apoptotic ROS effects, the adipocytes, in turn, ectopically activate the genetic program of melanogenesis, thus neutralizing an excess of ROS. Adipocytic melanin may also suppress the secretion of pro-inflammatory molecules, thereby decreasing the proinflammatory background in the obese body and alleviating the metabolic syndrome. Very high levels of polymorphism in the human genes regulating melanin biosynthesis provide a basis for the highly individual melanogenic response of adipocytes that may account for the differences in an individual's propensity to develop secondary complications of obesity.

The main derivation that follows from this hypothesis is that tmelanin production is linked to suppression of the pro-inflammatory effects of accumulated visceral fat. The potential impacts of this idea could be significant, as the molecular compounds stimulating melanin biosynthesis are readily available for trials aimed at control of metabolic syndrome components. The intact and inducible melanin biosynthesis pathway in human adipose serves to suppress systemic inflammation associated with obesity. Stimulation of melanogenesis with a synthetic analogue of α-MSH is expected to delay the development of secondary complications of metabolic syndrome as an increase in the melanin content of adipocytes shown to be paralleled by suppression of oxidative stress {deduced from the observed decrease in the levels of oxidative stress biomarkers and pro-inflammatory molecules (FIG. 1 and FIG. 2)}.

The obvious candidates for the regulatory molecules responsible for regulation of the adipocytic melanogenesis are α-melanocyte stimulating hormone (α-MSH), which is intimately connected both to melanogenesis and to energy homeostasis, and its antagonist, melanin-concentrating hormone (MCH). Importantly, the melanocortin system is extensively implicated in the regulation of energy homeostasis and the control of metabolism. A major component of the melanocortin system, α-MSH, is produced by proteolysis of pro-opiomelanocortin (POMC) precursor peptide and serves as a feeding suppressor. Genetic defects inactivating the receptors for α-MSH have been shown to produce obesity in humans and in experimental animals [Farooqi I S et al., 2003]. MCH, an antagonist of α-MSH, is an appetite-stimulating cyclic neuropeptide encoded by gene PMCH. Both peptides are produced in the hypothalamus and act centrally. The level of central nervous melanocortin system activity potently and rapidly influences the balance among cellular glucose uptake, triglyceride synthesis, lipid deposition, and lipid mobilization in liver, muscle, and adipose tissue [Nogueiras et al., 2007]. In addition, α-MSH and MCH peptides are produced in various peripheral tissues including adipose.

Both α-MSH and MCH exert a profound autocrine and paracrine effects. α-MSH and its synthetic analogs have been studied in rodent models of obesity, with incompletely understood results. In the CNS, α-MSH appears to increase sensitivity to insulin, while α-MSH in the periphery seems necessary for insulin resistance [Costa J L et al, 2006; Brennan M B et al., 2003]. Among other α-MSH dependent peripheral effects relevant to metabolic syndrome are the decrease in insulin secretion by the pancreatic β-cell [Shimizu H et al, 1995] and stimulation of lipolysis [Cho K J et al., 2005].

In adipose tissue, α-MSH increases leptin release without altering its synthesis [Mastronardi et al., 2005]. In turn, leptin increases the release of α-MSH into the circulation, suggesting a possible feedback loop between the sites of α-MSH release and the release of leptin from the adipose tissue [Hoggard et al., 2004]. The physiological significance of this putative feedback loop probably depends upon the underlying state of energy balance, because low plasma levels of α-MSH in fasting animals are paralleled by low plasma leptin [Hoggard et al., 2004].

Interestingly, in one human study, an intranasal administration of a -MSH to lean healthy volunteers resulted in a distinct reduction of body weight and body fat that was accompanied by significant decreases in leptin and insulin plasma concentrations [Fehm H L et al., 2001]. Contrasting with normal-weight humans, overweight subjects were not susceptible to the weight reducing effects of α-MSH administration [Hallschmid M. et al, 2006], probably due to resistance. We believe that administration of α-MSH or its analogues will act to prevent or delay the onset of the secondary complications of obesity, rather than influence BMI or total amount of adipose accumulation.

Indeed, studies in cellular and animal models showed that α-MSH exerts potent protective and anti-inflammatory effects on cells of the immune system and on peripheral non-immune cell types expressing melanocortin receptors. At the molecular level, α-MSH affects various pathways implicated in regulation of inflammation and protection, i.e. nuclear factor kappaB activation, expression of adhesion molecules and chemokine receptors, production of proinflammatory cytokines and mediators, IL-10 synthesis, T cell proliferation and activity, inflammatory cell migration, expression of antioxidative enzymes, collagen deposition, and apoptosis [Brzoska T et al., 2008; Bohm M et al., 2004]. The anti-inflammatory effects of α-MSH have been validated in animal models of experimentally induced fever and organ injury, irritant and allergic contact dermatitis, vasculitis, and fibrosis; ocular, gastrointestinal, brain, and allergic airway inflammation; and arthritis [Brzoska T et al., 2008; Colombo G et al., 2005; Lee T H et al. 2006].

It is likely that the anti-inflammatory effect of α-MSH-like compounds can be employed for curtailing sequelae of obesity and overweight. Importantly, molecular compounds that stimulate melanin biosynthesis by mimicking the effects of α-MSH are readily available for trials aimed at control of metabolic syndrome components. Synthetic agonists of α-MSH receptors, melanotan II and bremelanotide, have already been proven safe in human trials for therapeutic tanning and some non-obesity-related diseases. We propose Melanotan II as one of the possible preventive medication that may curtail development of sequelae of adipose accumulation in an animal model of dietary obesity.

Studies of serum levels of α-MSH and MCH in humans are scarce. An early study involving obese and lean females did not reveal any correlation between BMI and levels of α-MSH in plasma [Nam S Y et al., 2001]. Another, more recent, study showed that plasma levels of α-MSH are significantly elevated in obese humans, and that this elevation correlates to fat mass (R=0.586, P<0.001) and leptin levels (R=0.41, P<0.05) [Hoggard N. et al., 2004]. Other studies failed to demonstrate any significant correlation between α-MSH levels and any parameter of adiposity or diet composition [Donahoo W T et al., 2009; Gavrila et al., 2005], but showed that serum MCH levels are independently and positively associated with body mass index and fat mass [Gavrila et al., 2005]. Both quantitative RT-PCR and immunohistochemistry demonstrated notable expression of α-MSH receptor MC1-R in human adipocytes and adjacent macrophages [Hoch M et al., 2007]. Interestingly, in subcutaneous fat the expression of MC1-R mRNA was increased in obese patients as compared to controls [Hoch M. et al., 2007].

As extra cutaneous production of melanin in adipose has been discovered only recently, in our own work (Randhawa et al., 2008), there have been no attempts to connect the systemic anti-inflammatory properties of a-MSH to its melanogenic effects. In humans, no attempts to link inflammation and changes in a-MSH levels have been reported so far. However, it was observed that plasma a-MSH levels are low in samples collected from patients with septic shock at the beginning of septicemia, and that these levels gradually increase in patients who recover but not in those who die [Catania A et al., 2000]. Though intriguing, these results could not provide any insight into the role of a-MSH in the development of chronic conditions, as septicemia is a relatively acute event.

The invention described here relies on the notion that extra cutaneous melanin production is linked to suppression of the pro-inflammatory effects of accumulated visceral fat. We postulate that the molecular compounds stimulating melanin biosynthesis could be used to control systemic inflammation and other consequences of metabolic syndrome and should be tested in clinical trials in obese and overweight populations. Besides the potential for immediate clinical application that would dramatically improve the health of a large portion of the U.S. population, this project possesses a substantial basic science value, as it deals with an entirely novel phenomenon of melanin biosynthesis in adipose. Additionally, other populations exposed to high levels of oxidative stress could be also preventively treated with a-MSH, melanotan II and its analogues. Namely, these populations comprise of chronic cigarette smokers, individuals with depressive disorders or depressive episodes, and other individuals under any environmental stress.

### BRIEF DESCRIPTION OF THE DRAWINGS

FIG. 1. In the absence of melanin, dietary factors such as free fatty acids (FFA) and high levels of glucose in the bloodstream generate excess ROS in adipocytes, which in turn recruit macrophages to adipose tissue. The combined effect is the increased release of pro-inflammatory cytokines and decreased release of anti-inflammatory cytokines.

FIG. 2. The production of melanin by adipocytes may serve to sequester excess ROS, thereby inhibiting the release of pro-inflammatory cytokines and stimulating the release of anti-inflammatory cytokines.

FIG. 3. qRT-PCR comparison of expression levels of TYR, TYRP1, and DCT mRNAs in adipose samples. Red: morbidly obese. Blue: lean.

FIG. 4. Relative abundance of MC1R, TYR, TYRP1, and TYRP2 transcripts in adipose samples collected from morbidly obese (OA, n=5) and non-obese (NOA, n=2) subjects.

FIG. 5. In situ hybridization of a human TYR RNA probe on sections of visceral adipose tissues. A and B: T7 (cTYR) probe, morbidly obese subjects; C: T7 (cTYR) probe, lean subject; D: SP6 (control) probe, obese subject. TYRP1 and DCT probes demonstrated similar results (not shown).

FIG. 6. Immunohistochemical staining of visceral adipose tissue sections from morbidly obese and non-obese subjects for human TYR protein (20×). Red: TYR staining. Blue: DAPI (nuclei). A: morbidly obese subject. B: lean subject. G and H: Negative control (secondary antibodies) and DAPI.

FIG. 7. Fontana-Masson stain of human adipose tissue (20×). A. Adipose form obese subject. B. Adipose from lean subject

FIG. 8. Cell debris sediments from adipose tissue samples from morbidly obese subjects contain visible amounts of black pigment.

FIG. 9. LC-MS multi-ion SIM chromatogram of PTCA peak.

FIG. 10. Results of the L-[U-14C] tyrosine assay. OA1-7: adipose samples from morbidly obese individuals. NOA: adipose sample from non-obese subject; Ga: gastric sample; Liv: liver sample; MNT1: positive control (on a different scale).

FIG. 11. Adiponectin serum concentrations and its mRNA levels in omental adipose tissue of patients with and without insulin resistance

FIG. 12. An analysis of gene expression patterns in adipose by Ingenuity IPA 4.0 highlighted a network centered on IL-6 and TNF- as the most important for pathogenesis of NASH (A), while genes regulated by TGF-signal (B) were identified as participating in the development of the primary phenotype of the morbid obesity, but not involved in its secondary complications. alpha analysis of the Adiponectin serum concentrations and mRNA levels in omental adipose tissue of patients with and without insulin resistance.

FIG. 13. A comparison of serum concentrations of TNF-α in various cohorts of patients. Note that TNF-α levels are lower in morbidly obese patients with normal livers (Control I) as compared to lean blood donors (Control II) (P<0.0001).

FIG. 14. Results of the L-[U-14C] tyrosine assay of pooled adipose samples of C57B1/6 mice on Regular and High-Fat diets

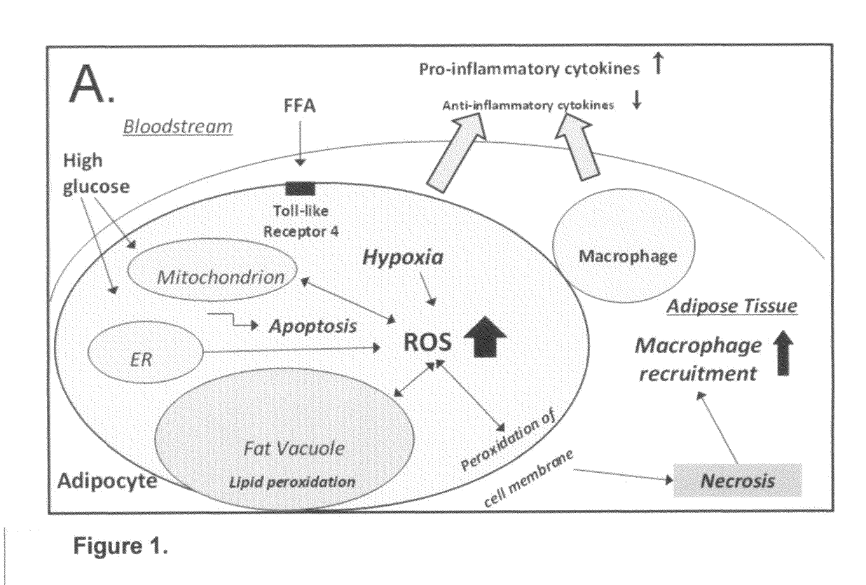

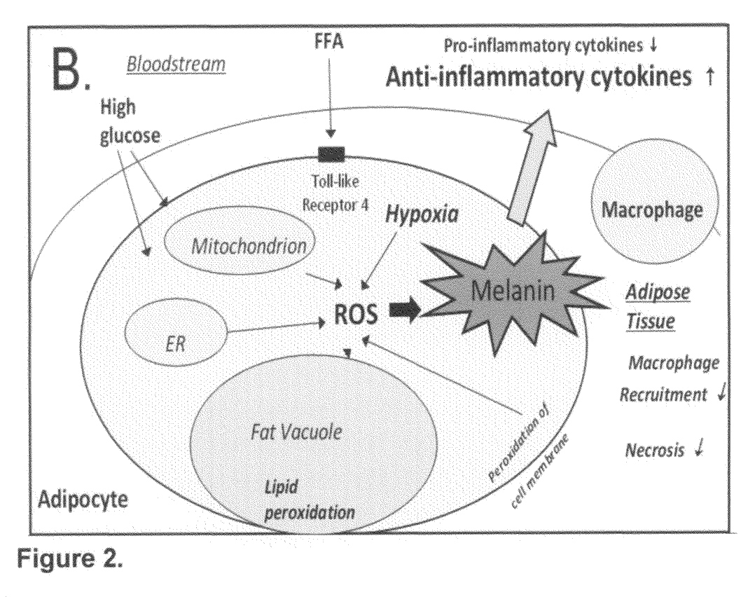

Table 1. C14 assay readouts (CPM values) of the total outputs of the melanogenic pathway in extracts of primary human adipocytes differentiated in culture 14 days and 21 days as compared to extracts of adipose samples from morbidly obese patients.

### EXAMPLES

In humans, melanin is produced in melanocytes, in retinal pigment epithelium (RPE) cells and in cells of the inner ear and the central nervous system. Our recent study demonstrated for the first time that the melanin biosynthesis pathway is functional in adipose tissue (Randhawa et al., 2009).

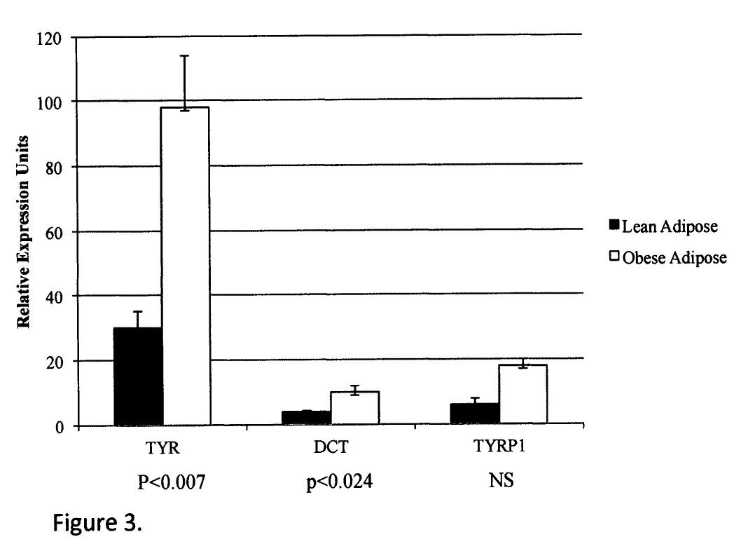

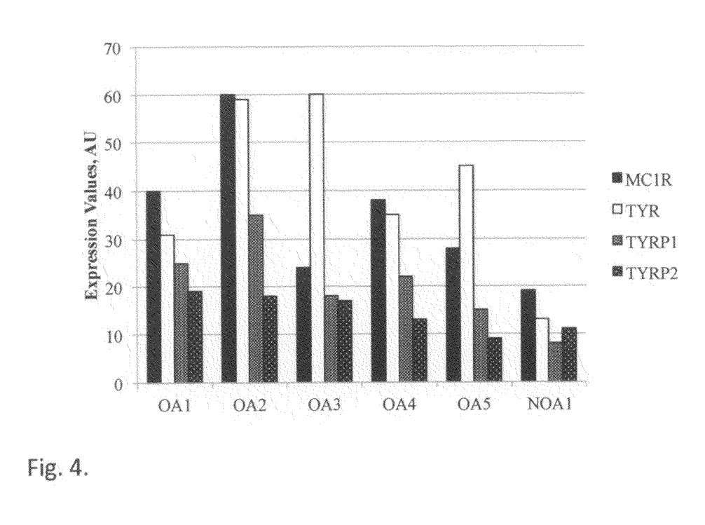

Over-expression of melanogenesis-related genes in adipose of morbidly obese subjects Over-expression of melanin biosynthesis-related genes in adipose of morbidly obese subjects was noted during analysis of cDNA microarray experiments comparing mRNA profiles in visceral adipose samples of 50 morbidly obese patients undergoing bariatric surgery and 9 nonobese organ donors regarded as controls (Baranova et al., 2005). Statistically significant over-expression of melanogenic genes encoding tyrosinase related protein 1 (TYRP1), dopachrome tautomerase (DCT/TYRP2), melanosome transport protein RAB27a, and Melan-A (MLANA) has been detected (P<0.05) (Baranova et al., 2005). The probe for the tyrosinase encoding gene TYR was not present at the microarray slides. Real-Time SYBR Green PCR analysis of mRNA samples from 6 obese and 3 non-obese subjects has been performed using 18S RNA as an internal control. Expression levels were significantly different for TYR (P<0.007) and DCT/TYRP2 (P<0.024). Expression levels of TYRP1 demonstrated a nonsignificant upward trend in samples from obese subjects (see FIG. 3). Quantification of mRNAs for TYR, TYRP1, TYRP2/DCT, and α-MSH receptor MC1R in 7 additional samples demonstrated similar results (FIG. 4). Gastric mRNAs were used as a negative control (not shown).

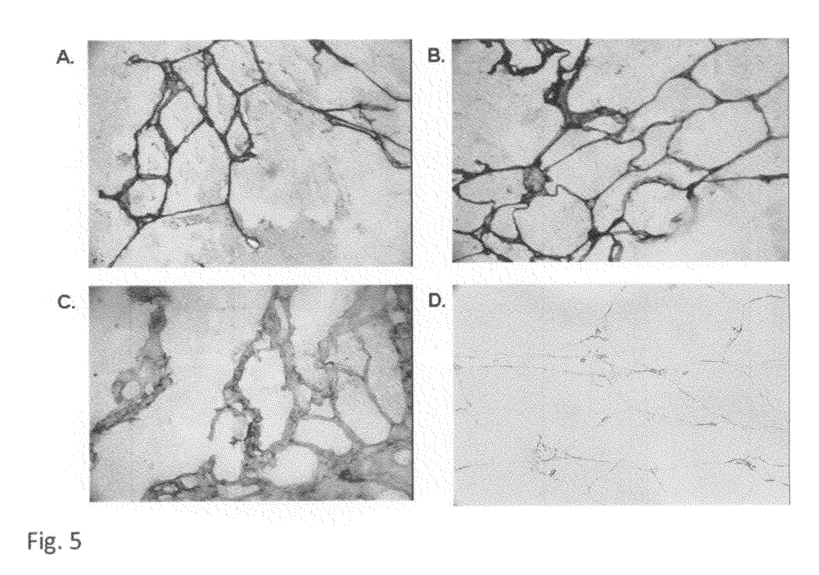

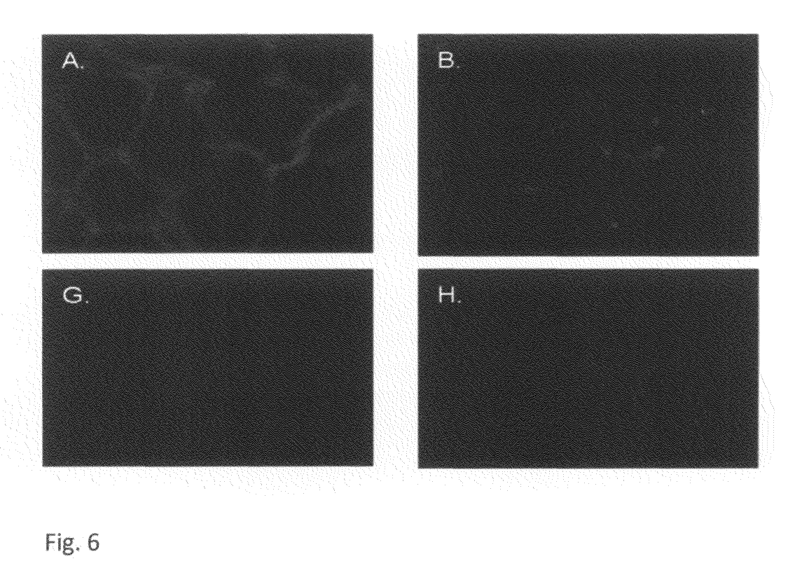

Adipocyte-specific expression of tyrosinase was confirmed by in situ hybridization on sections of visceral adipose tissue from morbidly obese and non-obese subjects (FIG. 5) and by immunohistochemistry (FIG. 6). TYRP1 and DCT probes demonstrated similar results (not shown). Therefore, all of the methods of detection indicated over-expression of the genes facilitating the biosynthesis of the melanin.

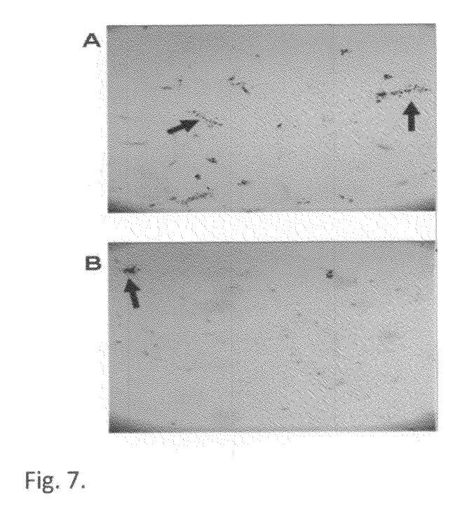

Fontana-Masson staining of human adipose samples showed an accumulation of black pigment in the periphery of adipocytes in morbidly obese patients (FIG. 7). Little black granules were detected in the adipose tissue of non-obese subjects (FIG. 7). The aforementioned staining was specific to adipocytes, as microvessels and stromal fibroblasts were negative. In adipocytes, the staining pattern reflected the cytoplasmic location of melanin granules, as the central vacuoles filled with lipids remained unstained (FIG. 7).

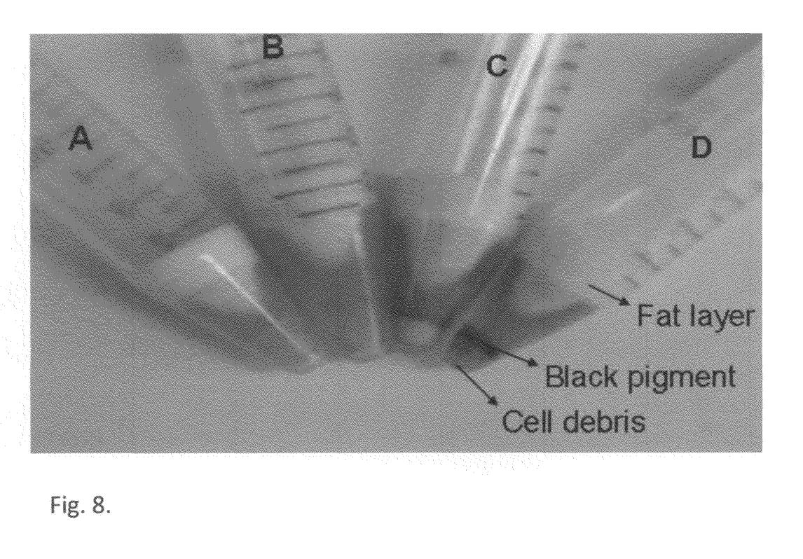

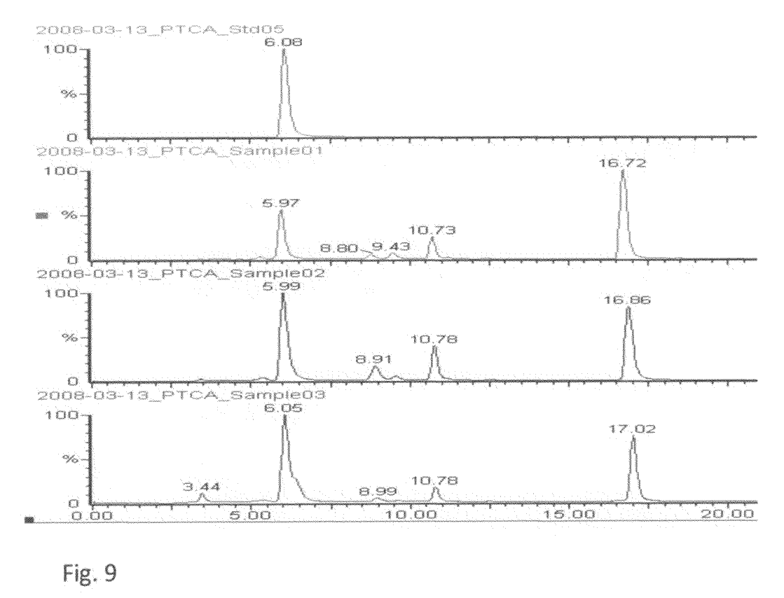

To determine whether the black pigment present in adipose tissue is eumelanin rather than lipofuscin or some other type of non-melanin pigment, we developed an LC-UV-MS assay that detects pyrrole-2,3,5-tricarboxylic acid (PTCA), the most characteristic degradation product of eumelanin. Synthetic melanin and melanin extracted from human hair were used as positive controls, and HL-60 human leukemia cells were used as a negative control. Samples of adipose tissue were liquefied by sonication; the resulting pellets were dissolved in NaOH, then subjected to permanganate oxidation and used for PTCA quantification by LC-UV-MS. Even at the stage of homogenization, there were notable differences in pigmentation between adipose tissue extracts of non-obese and obese adipose samples (FIG. 8). LC-UV-MS analysis positively identified PTCA (see FIG. 9 for LC-MS SIM chromatogram at 6 min; negative ESI mass spectra of PTCA peak and UV/VIS chromatograms at 270 nm demonstrated similar results that are not shown).

The collisional activated dissociation of the PTCA precursor ion with a mass-to-charge ratio (m/z) of 198 produces abundant product ions at m/z 154 and 110. Ion with m/z 154 was used for quantitation, as it does not co-elute with an interfering component of the extract. Quantification of the PTCA ion 154 m/z in the profiled visceral adipose sediments of three morbidly obese subjects revealed the presence of the ion in concentrations ranging from 0.19 to 0.12 ng/μL, while its concentrations in oxidized adipose sediment from two non-obese subjects were 0.05 ng/μL (abdominal visceral adipose) and 0.0009 ng/μL (perirenal fat). In the samples of gastric tissue and HL-60 cells (negative controls), the aforementioned PTCA ion was not detected.

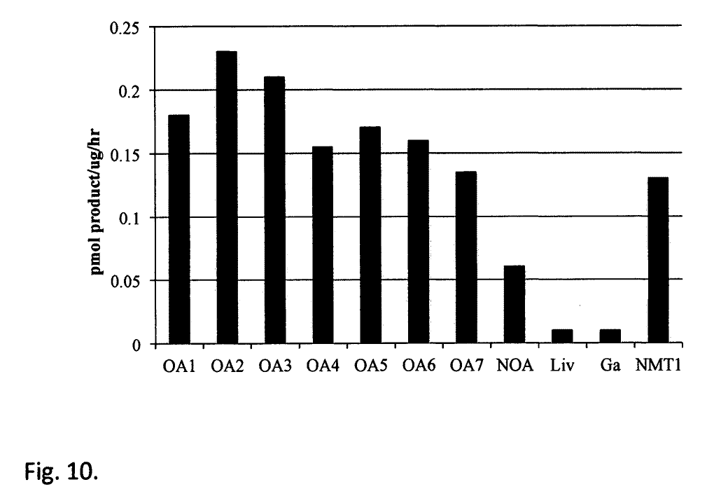

The total output of the melanogenic pathway was quantitatively evaluated by incorporation of labeled L-[U-14C] tyrosine into its final product, acid-insoluble melanin. Melanogenic activities in the liver and the gastric controls were similar to the blank negative control with no protein extract added, while activities in the adipose tissue samples of all 7 obese subjects were much higher (FIG. 10) and were characterized by marked heterogeneity. Activity in the adipose tissue sample of the non-obese individual was less than one-half of that of the obese subjects (obese subjects: 0.17+/−0.03 pmol product/μg/hr; lean subject: 0.05 pmol product/μg/hr).

Currently it is unknown whether the extent of melanin biosynthesis in the adipose could be negatively correlated with patient morbidity. Most of the adipose tissue donors enrolled in our study had an underlying disease of the non-alcoholic fatty liver disease (NAFLD) spectrum. Interestingly, the morbidly obese subject with the highest melanin levels by LC/UV/MS assay (0.42 ng/μL) had a normal liver biopsy. The respective sample of fat was taken at a time of bariatric surgery reversal, when patient had an almost-normal BMI of 26. This finding may indicate that the melanin accumulated in obese individuals remains in the adipose despite a loss of weight. Both patients diagnosed with NASH had substantially lower melanin contents in their adipose, 0.16 ng/μL and 0.18 ng/μL, respectively). Interestingly, the limited clinical information available for 5 out of the 8 obese patients sampled for visceral adipose tissue revealed a positive correlation between fasting glucose levels and total outputs of the melanogenic pathway in adipose tissues (R=0.9685, p<=0.007). This observation might indicate a compensatory activation of adipocytic melanogenesis pathway and insulin resistance.

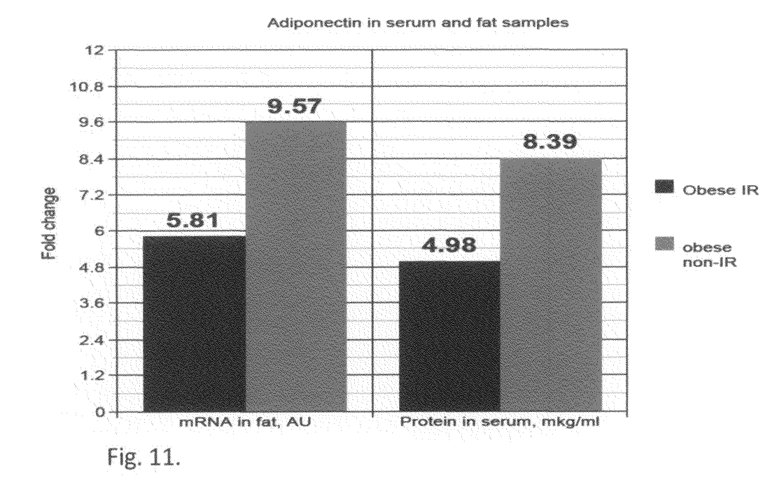

Adipose tissue is an active endocrine organ that secretes a variety of metabolically important substances including adipokines. These factors affect insulin sensitivity and may represent a link between obesity, insulin resistance, and NAFLD. In 2006, we quantified mRNAs encoding adiponectin, leptin, and resistin in snap-frozen samples of intra-abdominal adipose tissue and respective protein levels in the serum samples of morbidly obese patients undergoing bariatric surgery (Baranova et al., 2006). The study design allowed us to exclude important confounders such as obesity, age, and gender. Patients were classified into two groups: Group A (with insulin resistance, N=11) and Group B (without insulin resistance, N=10). Adiponectin mRNA in intra-abdominal adipose tissue and serum adiponectin levels were significantly lower in Group A than in Group B (P<0.016 and P<0.03, respectively) (FIG. 11). Although serum resistin was higher in Group A than in Group B patients (P<0.005), resistin gene expression was not different between the two groups. Finally, for leptin, neither serum level nor gene expression was different between the two groups. Decreased adiponectin level was the only predictor of NASH in this study (P=0.024) (Baranova et al., 2006). Antioxidant properties of adiponectin are well-know (see Plant S. et al., 2008 as example); these observations prove that anti-oxidative resistance of adipose in insulin resistant obese individuals is lowered, in part, due to decreased levels of adiponecting production.

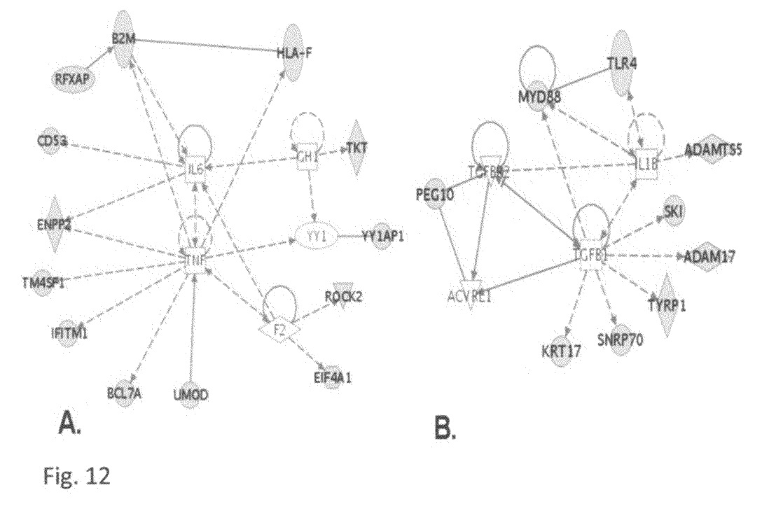

In 2007, we completed a paralleled microarray study of the gene expression profiles in liver and adipose samples collected from 27 morbidly obese patients with NASH, 7 morbidly obese controls with normal livers, and 6 non-obese controls (FIG. 12). In the NASH cohort as compared to obese controls, functional analysis identified prominent adipose-specific deregulation of genes encoding soluble pro-inflammatory molecules. Using Ingenuity IPA 4.0 software, we found that the adipose-specific TNF-_/IL-6 centered network was highlighted as the most important for the pathogenesis of NASH, while genes regulated by TGF-β signal were identified as participating in the development of the primary phenotype of the morbid obesity, but not involved in its secondary complications (FIG. 12). In the liver, a compensatory increase in the process of hepatic detoxification and a decrease in the genes comprising the network controlled by transcription factor COUP-TFII were noted. Our findings support the hypothesis that deranged adipocytic secretion plays an important role in the progression of NAFLD (Baranova et al.,

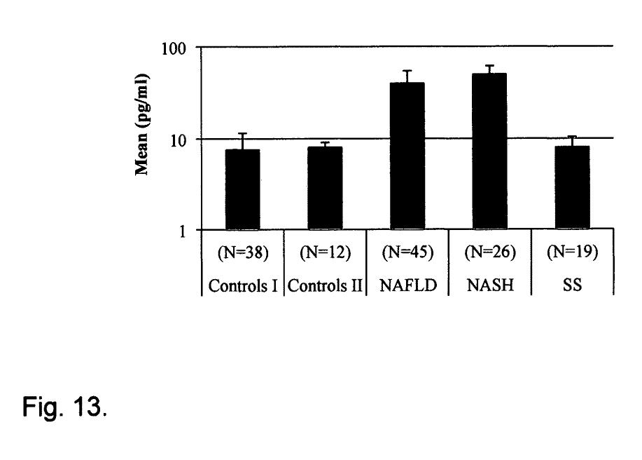

In 2008, we profiled fasting serum levels of insulin, glucose, visfatin, resistin, adiponectin, TNF-α, interleukin-8 (IL-8) and IL-6 in a cohort of 95 patients (26 NASH, 19 simple steatosis [SS], 38 obese controls with normal liver histology, and 12 non-obese controls). Among other findings, serum TNF-_{—} and IL-8 were higher in NAFLD patients when compared with both obese and non-obese controls. There was a significant correlation between serum TNFa and IL-8 (P<6 e-08), and between IL-6 and IL-8 (P<5 e-15). TNF-α levels demonstrated a significant increase from obese controls to simple steatosis and to NASH, thus emphasizing the potential role of TNF-α in the progression of NAFLD. Additionally, multivariate analysis identified TNF-α a level as an independent predictor of fibrosis in NASH patients (P<0.0004) (Jarrar et al., 2008). Interestingly, TNF-α levels were lower in morbidly obese patients with normal liver histology (Controls I, n=38, 1.91+/−0.25 pg/ml) than in lean blood donors (Controls II, n=12,2.3+/−0.39) (P<0.0001) (FIG. 13). These findings support the hypothesis that in a subset of morbidly obese patients adipose remains relatively inert, sustaining low levels of secretion and preventing the development of metabolic syndrome. We believe that induction of melanin biosynthesis may turn active, pro-inflammatory adipose into inert one, by providing ample amounts of melanin that will serve as a dump for reactive oxygen species and other damaging substances produced during lipid peroxidation.

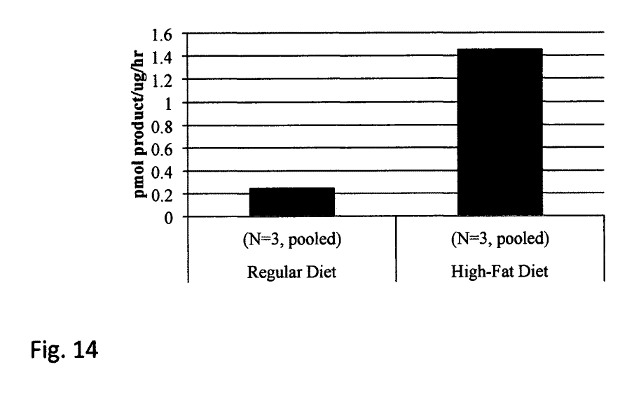

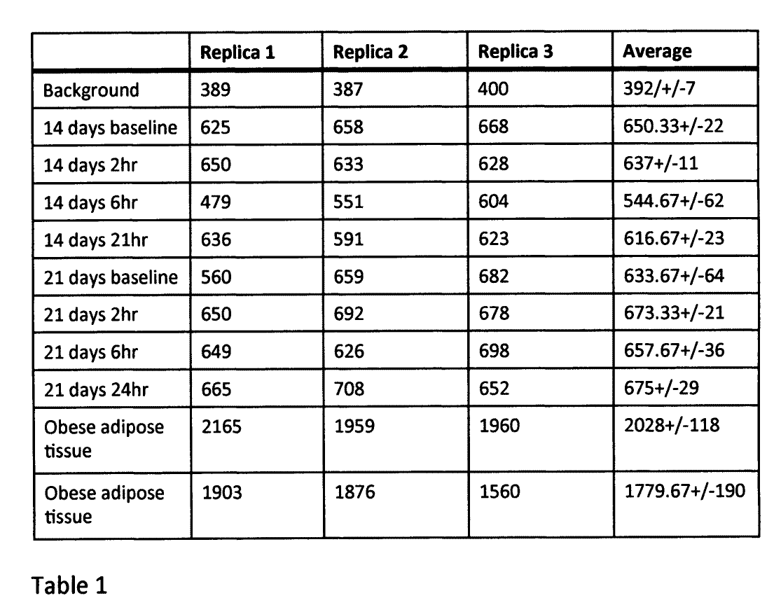

To ensure that the observed increase in melanin biosynthesis in the adipose tissue of morbidly obese subjects is not a unique characteristic of human adipose, we performed an L-[U-14C] tyrosine assay on the adipose samples collected from C57B1/6 animals fed 6 weeks of high fat diet (n=3) and of regular diet (n=3). Samples were provided by Dr. Gavrilova (Mouse Metabolism Core, NIDDK). Due to the small size of the adipose compartment in mice, high-fat diet and regular diet samples were profiled in pools. Liver tissue samples collected from the same animals and the MNT1 melanoma cells were used as negative and positive controls, respectively. The results obtained in this experiment were similar to those of the experiment with human samples described in above, namely, mice on high-fat diet systhesized substantiatlly larger amounts of melanin in their adipose as compared to mice on a regular diet (FIG. 14). The melanogenic outputs of the liver controls were similar to the blank negative control with no protein extract added. This experiment demonstrates feasibility of the preclinical studies of the melanin biosynthesis in adipose tissue in the mouse model.

What is claimed?

1. A method for treating a subject to abate inflammation by employing αMSH analogs to stimulate melanogenesis in adipose tissue.

2. A method according to claim 1 where the subject is a person and where treating the inflammation delays the development of complications in metabolic syndromes.

3. A method according to claim 1 of αMSH analog.

4. A method for treating non-communicable age-related diseases (NCDs), in a subject, such as heart disease, cancer, stroke, type 2 diabetes and chronic lower respiratory diseases smokers and depression afflicted patients/subjects, the method comprising administering to a subject a therapeutically effective amount of a αMSH peptide

5. A method according to claim 4 where the peptide is an analog

6. A method according to claim 4 where the peptide is alpha-melanocyte stimulating hormone-related tripeptide K(D)P

7. A method according to claim 4 where modulating inflammatory cytokines and growth factors such as TNFα, IL6, Insulinγ/IGF-1

8. A method of abrogating oxidative stress through stimulation of melanogenesis using αMSH analogs

9. A method for down regulation of reactive oxygen species

10. The method according to claim 4, further comprising the step of selecting a subject having depression, chronic smokers and/or obese individual

11. The method according to claim 4 wherein the treatment results in decrease of inflammation

12. The method according to claim 1 wherein the subject is a mammal.

13. The method according to claim 1 wherein the mammal is a human

14. The method according to claim 1 wherein the mammal is a domesticated animal.

15. The method according to claim 4 wherein the peptide is administered orally, sublingually, through transdermal delivery or subcutaneously

16. The method according to claim 4 wherein the peptide is made synthetically or produced in bacteria and extracted (recombinant) or produced in bacteria and applied orally as part of bacterial preparation (probiotic)
